(19) United States
(12) Patent Application Publication
(10) Pub. No.: US 2004/0077521 A1
(43) Pub. Date: Apr. 22, 2004

(54) Method of inducing relaxation and sleep

(76) Inventor: Alana Dyanne, Redmond, WA (US)

Correspondence Address: PATRICK M. DWYER PC, 1818 WESTLAKE AVENUE N, SUITE 114, SEATTLE, WA, 98109, US

(21) Appl. No.: 10/316,947

(22) Filed: Dec. 10, 2002

Related U.S. Application Data
(60) Provisional application No. 60/338,967, filed on Dec. 10, 2001.

(51) Int. Cl. A61K 31/00; A61K 35/78; A61H 23/00

(52) U.S. Cl. 514/1; 601/107; 424/745; 424/750; 424/764

(57) ABSTRACT

A method of inducing relaxation or sleep comprising applying a drop of herbal formula to at least two of a group of twelve body locations, such as two points each on the inside of each wrist two inches above wrist crease, the center of the back of the head in a large hollow under the base of the skull, below the base of the skull, on the two large vertical neck muscles to either side of the spine, along large bone behind each ear and downward, and at the temples at the edge of the hairline.

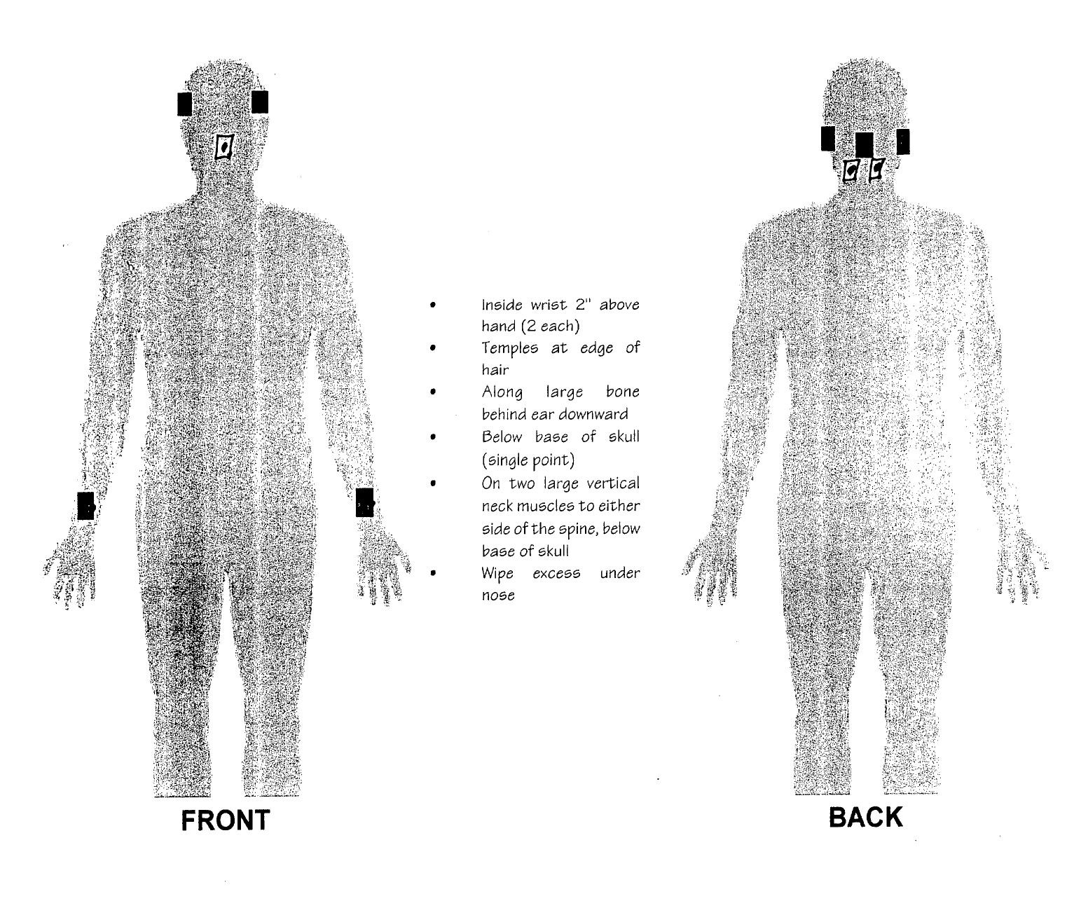

This application claims priority to U.S. Provisional Patent Ser. No. 60/338,967 filed Dec. 10, 2001.

Sleep deprivation has been called the ‘silent epidemic’, affecting more than 100 million American adults and 60% of all school age children. Current annual business losses are estimated to be $150 billion. AAA attributes 100,000+car ‘crashes’ per year due to ‘falling asleep at the wheel’. Several major worldwide disasters have been attributed to sleep deprivation.

Until now options for sleep aids have been primarily limited to oral prescription and over-the-counter drugs which have many unwanted side effects including dependency, drowsiness upon awakening, liver toxicity, and brain fog.

I have combined holistic aromatic technology and Chinese acupressure techniques to successfully induce deep relaxation and promote restful sleep without side effects in a number of test studies.

Best results have so far been obtained by using a particular formulation called Dream Drops by Quiet Nite (14150 NE 20^{th} Street, Suite 155, Bellevue, Wash. 98007) and applying a small drop (or a short swipe from a roll-on container) of this formulation to each of two or more (and preferably all 12) of 12 selected acupressure points as set forth below. Alternatively, good results may be obtained by using a combination of one or more generally known relaxing oils and botanicals such as lavender, sweet marjoram, vetiver, and chamomile, all in any well know base that is readily absorbed through the skin. These alternative combinations can either be commercially available, or home made.

Selected points of application for the drops are identified below by name and number according to generally accepted acupressure charts; selected application points are also specified in the attached diagram.

Application is as follows:

1) Two points each on the inside of each wrist 2 inches above wrist crease (Inner Gate P 6 and Spirit gate H 7);

2) In the center of the back of the head in a large hollow under the base of the skull (Wind Mansion GV 16);

3) Below the base of the skull, on the two large vertical neck muscles to either side of the spine (Heavenly Pillar B 10);

4) Along large bone behind each ear and downward (Gates of Consciousness GB 20);

5) At the temples at the edge of the hairline (Pulse AB);

6) Wipe excess under nose (Middle of a Person GV 26).

I claim:

1. A method of inducing relaxation or sleep comprising applying a drop of the disclosed formula to at least two of a group of twelve points consisting of: two points each on the inside of each wrist 2 inches above wrist crease (Inner Gate P 6 and Spirit gate H 7); the center of the back of the head in a large hollow under the base of the skull (Wind Mansion GV 16); below the base of the skull, on the two large vertical neck muscles to either side of the spine (Heavenly Pillar B 10); along large bone behind each ear and downward (Gates of Consciousness GB 20); and at the temples at the edge of the hairline (Pulse AB); with the excess optionally wiped under the nose (Middle of a Person GV 26).

2. A method of inducing relaxation or sleep comprising applying a drop of the disclosed formula to at least two of a group of twelve points consisting of: the inside of wrist at 2″ above hand (2 each), at temples at edge of hair, along large bone behind ear downward, below the base of skull (single point), on two large vertical neck muscles to either side of the spine, and below base of skull.
